(19) United States
(12) Patent Application Publication
(10) Pub. No.: US 2020/0018737 A1
(43) Pub. Date: Jan. 16, 2020

(54) METHOD FOR DETECTING COMPROMISED ZONE IN CLEANROOM

(71) Applicant: L&K ENGINEERING (SUZHOU) CO., LTD, Suzhou (CN)

(72) Inventor: Chengyu YANG, Taiwan (CN)

(73) Assignee: L&K ENGINEERING (SUZHOU) CO., LTD, Suzhou (CN)

(21) Appl. No.: 16/524,583

(22) Filed: Jul. 29, 2019

(30) Foreign Application Priority Data
Aug. 7, 2018 (CN) 201810892463.1

(51) Int. Cl. G01N 33/00; G01N 17/04; B01D 46/42; B01D 53/38; F24F 3/16

(52) U.S. Cl. CPC G01N 33/0027; G01N 17/04; B01D 46/42; B01D 2279/51; F24F 3/161; B01D 2258/06; B01D 53/38

(57) ABSTRACT

Method for detecting compromised zone in cleanroom, including the following steps: (1) cleanroom including air supply space, ceiling, clean space, elevated floor, and return air space that are arranged sequentially from top down, wherein ceiling is divided into plurality of air supply zones, elevated floor is divided into plurality of exhaust zones, air supply zones and exhaust zones are arranged vertically in one-to-one corresponding manner with cylindrical space formed between corresponding two, cleanroom further includes detection mechanisms corresponding, one-to-one, to cylindrical spaces, and detection mechanism includes corrosion test specimen and detection unit; and (2) monitoring electrical parameters of corrosion test specimen of each detection mechanisms, and determining, if electrical parameters of corrosion test specimen of one detection mechanism change continuously in trend, that cylindrical space corresponding to this detection mechanism is compromised zone.

### FIELD OF THE INVENTION

The present invention relates to the field of clean production, and specifically, to a method for detecting a compromised zone in a cleanroom.

### BACKGROUND OF THE INVENTION

Cleanroom refers to a space with good airtightness and control for parameters such as air cleanliness, temperature, humidity, pressure, noise, and the like. The development of cleanroom is closely related to modern industry and cutting-edge technologies. The requirements for environment in the precision machinery industry (e.g., processing for gyroscopes, micro bearings, etc.), semiconductor industry (e.g., large-scale integrated circuit production), and the like advance the development of cleanroom technologies. According to domestic statistics, the pass rate of MOS tube production in an environment with no requirements for cleanliness level is only 10%-15%, and 64 is only 2% for memories. At present, it is very common to use cleanrooms in precision machinery, semiconductor, aerospace, atomic energy, and other industries.

Cleanrooms according to the prior art comprise a clean space, a ceiling, and an elevated floor, wherein the ceiling is provided with a plurality of air supply zones, each of the air supply zones comprises at least one air supply mechanism, and the elevated floor is provided with a plurality of exhaust zones. The air supply mechanism is typically an FFU device or comprises an FFU device and a chemical filter. The FFU device functions to supply the air and filter large particles of pollutants, and the chemical filter functions to filter corrosive gases. When corrosive gases in the clean space are detected, a point monitoring method is typically used to monitor corrosive gases in the clean space in real time. Namely, a plurality of sampling points are arranged in the clean space, and then the collected gas is sent to an analytical instrument via a pipeline. The analytical instrument analyzes the gas to determine whether the gas comprises corrosive gases. If yes, operations of the machine at the position of the sampling point are promptly suspended to prevent products from being contaminated.

However, the above method has the following drawbacks: first, it is impossible to fully cover a cleanroom having a large area with sampling points due to limitations by the pipeline length, making it impossible to achieve full monitoring; second, each analytical instrument is connected to a plurality of different sampling points, and consequently, there is residual gas from the previous analysis in the analytical instrument, making it impossible to ensure the purity of the gas for the following analysis and leading to inaccurate monitoring; third, there is a limited number of pipes that each analytical instrument can be connected to, and consequently there is a limited number of connected sampling points, making it necessary to provide a plurality of analytical instrument, which leads to high costs as the analytical instruments are expensive. In summary, the point monitoring method is difficult to achieve full and accurate monitoring, and the cost is high.

### SUMMARY OF THE INVENTION

The objective of the present invention is to provide a method for detecting a compromised zone in a cleanroom, which can achieve full and accurate detection of corrosive gases and lower costs.

To achieve the above objective, the present invention employs the following technical solution: a method for detecting a compromised zone in a cleanroom, comprising the following steps:

(1) providing a cleanroom including an air supply space, a ceiling, a clean space, an elevated floor, and a return air space that are arranged sequentially from top down, wherein the ceiling is divided into a plurality of air supply zones, the elevated floor is divided into a plurality of exhaust zones, the air supply zones and the exhaust zones are arranged vertically in a one-to-one corresponding manner with a cylindrical space formed between the corresponding two, the cleanroom further comprises detection mechanisms corresponding, one-to-one, to the cylindrical spaces, and the detection mechanism comprises a corrosion test specimen in contact with a corrosive gas flowing through the corresponding cylindrical space and a detection unit for detecting electrical parameters of the corrosion test specimen; and

(2) monitoring electrical parameters of the corrosion test specimen of each of the detection mechanisms, and determining, if the electrical parameters of the corrosion test specimen of one detection mechanism change continuously in a trend, that the cylindrical space corresponding to this detection mechanism is a compromised zone.

In the above step (2), for example, when the real-time resistance of the corrosion test specimen undergoes a positive change relative to the initial resistance (i.e., the real-time resistance continues to increase), it is determined that the gas flow in contact with the to-be-measured resistor contains a corrosive gas. Since the corrosion test specimen has a long life, it can be used for a long period. For each time of monitoring, therefore, it is necessary to compare with the real-time resistance of the test specimen at the beginning of the test.

In the above technical solution, the corrosion test specimen is a to-be-measured resistor, and the detection unit is a resistance-measuring circuit.

In the above technical solution, the corrosion test specimen and the detection unit form a Wheatstone bridge resistance-measuring circuit, and the corrosion test specimen functions as the to-be-measured resistor of the Wheatstone bridge resistance-measuring circuit.

In the above technical solution, the corrosion test specimen and the detection unit form a voltammetric resistance-measuring circuit, and the corrosion test specimen functions as the to-be-measured resistor of the voltammetric resistance-measuring circuit.

In the text above, the corrosion test specimen is a conductor or semiconductor having a resistance, which will be corroded after contacting a corrosive gas, leading to a change to its own resistance. By making use of this phenomenon, the core of the present application is to detect whether a corrosive gas is present according to changes to the resistance of the corrosion test specimen detected by the detection unit.

The shape of the corrosion test specimen may be a sheet, a strip, a rod, or other shapes commonly used in the art, which is not limited in the present application.

In the above technical solution, the detection mechanism comprises at least one corrosion test specimen, and the detection mechanism is arranged in at least one of the air supply space, the ceiling, the clean space, the elevated floor, and the return air space. Namely, the detection mechanism corresponding to each of the cylindrical spaces may use one corrosion test specimen or a plurality of corrosion test specimens. The number and installation positions of corrosion test specimens used by detection mechanisms corresponding to different cylindrical spaces may be the same or different.

In the above technical solution, each of the air supply zones comprises at least one air supply mechanism. Namely, each air supply zone may comprise one air supply mechanism or may comprise a plurality of air supply mechanisms.

In the above technical solution, the air supply mechanism is an FFU device or the air supply mechanism comprises an FFU device and a chemical filter.

In the above technical solution, the corrosion test specimen of the detection mechanism is arranged on the ceiling, and the detection mechanism and the air supply mechanism share a single chip machine.

In the above technical solution, the detection unit of the detection mechanism is in wireless communication with the single chip machine thereof.

In the above technical solution, the corrosion test specimen is an iron specimen, a copper specimen, a silver specimen or a semiconductor. The semiconductor is, for example, silicon, germanium, gallium arsenide, and the like.

Because of the adoption of the above technical solution, the present invention has the following advantages relative to the prior art:

(1) In the method for detecting a compromised zone in a cleanroom according to the present invention, a corrosion test specimen and a detection unit are provided. The corrosion test specimen contacts a corrosive gas flowing through the cylindrical space, and the detection unit detects electrical parameters of the corrosion test specimen. According to changes to the electrical parameters of the corrosion test specimen, it is then determined whether the gas flow in contact with the corrosion test specimen contains a corrosive gas. If the gas flow in contact with the corrosion test specimen contains a corrosive gas, a prompt for attention may be provided or the monitoring of environmental conditions of the machine in the cylindrical space may be enhanced to prevent an additional amount of the corrosive gas from entering the clean space and prevent products on the machine from being contaminated. On one hand, the detect mechanisms may be provided in a great number as they are not limited by the pipeline, and each detect mechanism monitors one zone of the clean space, thereby achieving full detection through regional monitoring. On the other hand, different corrosion test specimens are in contact with different gas flows, the gas flows do not cross each other, and therefore, accurate detection can be achieved. Moreover, since circuits and single chip machines are used for detection, no expensive analytical instruments are required, which greatly lowers the monitoring cost;

(2) In the method for detecting a compromised zone in a cleanroom according to the present invention, the detection mechanism and the air supply mechanism share a single chip machine, which can greatly lower the cost; and

(3) In the method for detecting a compromised zone in a cleanroom according to the present invention, the corrosion test specimen is arranged at the gas flow input side of the air supply zone and the gas flow input side of the exhaust zone, which causes a corrosive gas to be attached, as much as possible and in a prompt manner, to the corrosion test specimen, thereby improving the detection efficiency.

### BRIEF DESCRIPTION OF THE DRAWINGS

FIG. 1 is a schematic structural diagram of a cleanroom disclosed by the present invention;

FIG. 2 is a schematic diagram of the principle of a Wheatstone bridge resistance-measuring circuit disclosed by the present invention;

FIG. 3 is a schematic diagram of the principle of a voltammetric resistance-measuring circuit disclosed by the present invention.

Wherein, 10. air supply space, 20. ceiling, 21. air supply mechanism; 30. clean space; 40. elevated floor; 50. return air space; 60. corrosion test specimen.

### DESCRIPTION OF THE SPECIFIC EMBODIMENTS

The present invention will be further described below with reference to the accompanying drawings and embodiments:

### Embodiment I

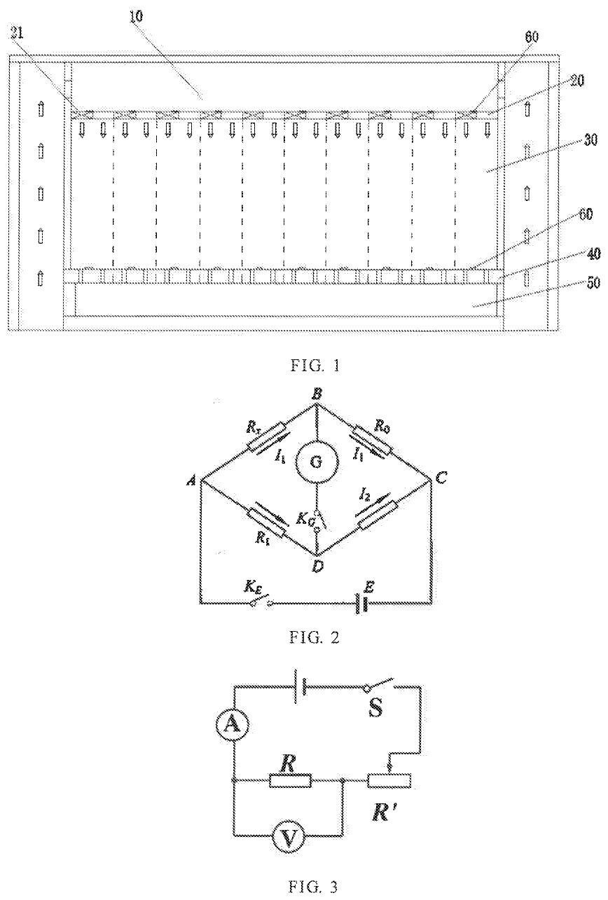

Referring to FIG. 1, as indicated by the legends therein, a method for detecting a compromised zone in a cleanroom comprises the following steps:

(1) providing a cleanroom including an air supply space 10, a ceiling 20, a clean space 30, an elevated floor 40, and a return air space 50 that are arranged sequentially from top down, wherein the ceiling 20 is divided into a plurality of air supply zones, the elevated floor 40 is divided into a plurality of exhaust zones, the air supply zones and the exhaust zones are arranged vertically in a one-to-one corresponding manner with a cylindrical space formed between the corresponding two, the cleanroom further comprises detection mechanisms corresponding, one-to-one, to the cylindrical spaces, and each detection mechanism comprises a corrosion test specimen 60 in contact with a corrosive gas flowing through the corresponding cylindrical space and a detection unit for detecting electrical parameters of the corrosion test specimen 60; and

(2) monitoring electrical parameters of the corrosion test specimen 60 of each of the detection mechanisms, and determining, if the electrical parameters of the corrosion test specimen 60 of one detection mechanism change continuously in a trend, that the cylindrical space corresponding to this detection mechanism is a compromised zone.

In the text above, the corrosion test specimen 60 is a to-be-measured resistor, and the detection unit is a resistance-measuring circuit. The corrosion test specimen 60 and the detection unit form a Wheatstone bridge resistance-measuring circuit, and the corrosion test specimen 60 functions as the to-be-measured resistor of the Wheatstone bridge resistance-measuring circuit.

Taking a Wheatstone balanced bridge as an example, the principle is shown in FIG. 2. Standard resistors R0, R1, and R2 and a to-be-measured resistor RX are connected to form a quadrilateral with each side referred to as an arm of the suspension bridge. A power source E is connected between the opposite angles A and C, and a galvanometer is connected between the opposite angles B and D. Therefore, the bridge consists of three parts, i.e., four arms, a power source and a galvanometer. When switches KE and KG are closed, each branch has a current flowing through, and the galvanometer branch functions to connect two branches of ABC and ADC, which is like a bridge and therefore, is referred to as a “bridge.” No current will flow through the bridge by appropriately adjusting the values of R0, R1, and R2, i.e., the current flowing through the galvanometer IG=0. At this moment, the electric potential is the same for point B and point D. Such a state of the bridge is referred to as the balanced state. At this moment, the potential difference between A and B is equal to the potential difference between A and D, and the potential difference between B and C is equal to the potential difference between D and C. Assuming that the current is I1 and I2 in the ABC branches, respectively, and according to the Ohm's law, it is obtained that

I1RX=I2R1;

I1R0=I2R2;

The first equation is divided by the equation to obtain

RX/R0=R1/R2 (1)

The equation (1) is referred to as the balance condition for a bridge, and it can be obtained from the equation (1) that

RX=(R1/R2)R0 (2)

The resistance value of the to-be-measured resistor may be calculated according to the equation (2).

In the text above, the air supply mechanism 21 is an FFU device or the air supply mechanism 21 comprises an FFU device and a chemical filter.

Preferably, detection mechanism comprises two corrosion test specimens arranged on the ceiling 20 and each of the air supply zones comprises one air supply mechanism 21. The corrosion test specimen of the detection mechanism is arranged on the ceiling, and the detection unit of the corrosion test specimen arranged on the ceiling 20 and the air supply mechanism 21 share a single chip machine. The corrosion test specimen 60 is an iron specimen, a copper specimen, a silver specimen or a semiconductor.

In practical use, operations of the air supply mechanism 21, adjustment of the detection unit, and computation of resistance of the to-be-measured resistor are controlled by the single chip machine. If a cylindrical space is a compromised zone, a prompt for attention is timely provided or the monitoring of environmental conditions of the machine in the compromised zone is enhanced.

### Embodiment II

It is the same as Embodiment I with the difference being that the corrosion test specimen 60 and the detection unit form a voltammetric resistance-measuring circuit, and the corrosion test specimen 60 functions as the to-be-measured resistor of the voltammetric resistance-measuring circuit. The principle is shown in FIG. 3.

Voltammetric resistance measuring is a common method to use an ammeter and a voltmeter to directly measure a to-be-measured resistor, and the resistance is measured through the Ohm's law for partial circuit: R=U/I. The ammeter is used to measure a current flowing through an unknown resistor under this voltage, and then the resistance of the unknown resistor is calculated, which is roughly divided into two types: internal connection of the ammeter and external connection of the ammeter. The so-called external connection and internal connection means that the ammeter is connected outside or inside the voltmeter, and the specific steps are as follows:

(1) adjusting the indicators of the ammeter A and the voltmeter V to zero, connecting an object according to a circuit diagram, and adjusting the resistance of a sliding rheostat R′ to the maximum;

(2) closing the switch S, adjusting the slider of the sliding rheostat R′ to an appropriate position, and taking the reading I of the ammeter A and the reading U of the voltmeter V, respectively; and

(3) calculating the value of R according to the equation R=U/I.

According to the method above, the slider of the sliding rheostat is adjusted to change the current in a to-be-measured resistor and the voltage at two ends, and multiple sets of R values are measured.

### Embodiment III

It is the same as either Embodiment I or Embodiment II with the difference being that the detection unit is an ohm gauge or a multimeter.

### Embodiment IV

It is the same as any one of Embodiments I to III with the difference being that each detection mechanism may further comprise one corrosion test specimen or two or more corrosion test specimens. The corrosion test specimens are arranged at any position in the air supply space, the ceiling, the clean space, the elevated floor, and the return air space.

### Embodiment V

It is the same as any one of Embodiments I to IV with the difference being that the detection unit of the detection mechanism is in wireless communication with the single chip machine thereof.

The above description of the disclosed embodiments enables those skilled in the art to implement or use the present invention. Various modifications of these embodiments would be obvious to those skilled in the art. The general principles defined herein may be implemented in other embodiments without departing from the spirit or scope of the present invention. Therefore, the present invention will not be limited to these embodiments illustrated herein, but will conform to the broadest scope consistent with the principles and novel features disclosed herein.

1. A method for detecting a compromised zone in a cleanroom, comprising the following steps:
(1) providing a cleanroom including an air supply space, a ceiling, a clean space, an elevated floor, and a return air space that are arranged sequentially from top down, wherein the ceiling is divided into a plurality of air supply zones, the elevated floor is divided into a plurality of exhaust zones, the air supply zones and the exhaust zones are arranged vertically in a one-to-one corresponding manner with a cylindrical space formed between the corresponding two, the cleanroom further comprises detection mechanisms corresponding, one-to-one, to the cylindrical spaces, and the detection mechanism comprises a corrosion test specimen in contact with a corrosive gas flowing through the corresponding cylindrical space and a detection unit for detecting electrical parameters of the corrosion test specimen; and
(2) monitoring electrical parameters of the corrosion test specimen of each of the detection mechanisms, and determining, if the electrical parameters of the corrosion test specimen of one detection mechanism change continuously in a trend, that the cylindrical space corresponding to this detection mechanism is a compromised zone.

2. The method for detecting a compromised zone in a cleanroom according to claim 1, characterized in that the corrosion test specimen is a to-be-measured resistor, and the detection unit is a resistance-measuring circuit.

3. The method for detecting a compromised zone in a cleanroom according to claim 2, characterized in that the corrosion test specimen and the detection unit form a Wheatstone bridge resistance-measuring circuit, and the corrosion test specimen functions as the to-be-measured resistor of the Wheatstone bridge resistance-measuring circuit.

4. The method for detecting a compromised zone in a cleanroom according to claim 2, characterized in that the corrosion test specimen and the detection unit form a voltammetric resistance-measuring circuit, and the corrosion test specimen functions as the to-be-measured resistor of the voltammetric resistance-measuring circuit.

5. The method for detecting a compromised zone in a cleanroom according to claim 1, characterized in that the detection mechanism comprises at least one corrosion test specimen, and the detection mechanism is arranged in at least one of the air supply space, the ceiling, the clean space, the elevated floor, and the return air space.

6. The method for detecting a compromised zone in a cleanroom according to claim 1, characterized in that each of the air supply zones comprises at least one air supply mechanism.

7. The method for detecting a compromised zone in a cleanroom according to claim 6, characterized in that the air supply mechanism is an FFU device or the air supply mechanism comprises an FFU device and a chemical filter.

8. The method for detecting a compromised zone in a cleanroom according to claim 6, characterized in that the corrosion test specimen of the detection mechanism is arranged on the ceiling, and the detection mechanism and the air supply mechanism share a single chip machine.

9. The method for detecting a compromised zone in a cleanroom according to claim 1, characterized in that the detection unit of the detection mechanism is in wireless communication with the single chip machine thereof.

10. The method for detecting a compromised zone in a cleanroom according to claim 1, characterized in that the corrosion test specimen is an iron specimen, a copper specimen, a silver specimen or a semiconductor.
